SEQUENCE LISTING (1 sequence)

SEQ ID NO: 1 - DNA, 28 bp, synthetic construct
  source 1..28: mol_type = other DNA
tttttggtggctggagggggcgcgaacg

(19) United States
(12) Patent Application Publication
(10) Pub. No.: US 2024/0000975 A1
(43) Pub. Date: Jan. 4, 2024

(54) BIOPROBE FOR NON-INVASIVE DIAGNOSIS OF PARKINSON'S DISEASE TRIGGERED BY INTESTINAL MICROENVIRONMENT, PREPARATION METHOD THEREFOR AND USE THEREOF

(71) Applicant: Sichuan Academy of Medical Sciences ° Sichuan Provincial People's Hospital, Chengdu (CN)

(72) Inventor: Yangbao MIAO, Chengdu (CN)

(73) Assignee: Sichuan Academy of Medical Sciences ° Sichuan Provincial People's Hospital, Chengdu (CN)

(21) Appl. No.: 17/985,169

(22) Filed: Nov. 11, 2022

(30) Foreign Application Priority Data
Jun. 30, 2022 (CN) 202210757668.5

(51) Int. Cl. A61K 49/00

(52) U.S. Cl. CPC A61K 49/0015; A61K 49/0065

(57) ABSTRACT

A bioprobe for a non-invasive diagnosis of Parkinson's disease triggered by an intestinal microenvironment, a preparation method thereof and use thereof are provided. The preparation method for the bioprobe provided by the present invention includes the following steps: (1) mixing europium nitrate with an organic ligand, and synthesizing a luminescent metal organic framework by a solvothermal method; (2) subjecting gold nanoparticles to a mixing reaction with aptamers to obtain Au-aptamer complexes; and (3) dissolving the luminescent metal organic framework in an Au-aptamer complex solution for a reaction, and washing the reaction product sequentially with deionized water, an ethanol solution, and a sodium dodecyl sulfate solution after the reaction to obtain the bioprobe. The present non-invasive oral bioprobe based on an intestinal microenvironment is configured for diagnosis of Parkinson's disease at an early stage.

### CROSS REFERENCE TO THE RELATED APPLICATIONS

This application is based upon and claims priority to Chinese Patent Application No: 202210757668.5, filed on Jun. 30, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC047_Sequence_Listing.xml, created on 09/30/2022, and is 1,988 bytes in size.

### TECHNICAL FIELD

The present invention relates to the technical field of biosensors, in particular to a bioprobe for non-invasive diagnosis of Parkinson's disease triggered by an intestinal microenvironment, a preparation method therefor and use thereof.

### BACKGROUND

Parkinson's disease is a chronic neurodegenerative disease that affects the central nervous system, primarily the motor nervous system. It is a common neurodegenerative disease, common in the elderly, with an average age of onset of about 60 years. Young-onset Parkinson's disease starting under the age of 40 is less common. It is characterized by the patient's motor stiffness or retardation and discovery of accumulation of Lewy bodies (LBs) in the brain. Lewy bodies are rich in α-synuclein (α-Syn) in an aggregated form. α-Syn is a 14 kDa protein with no well-defined structure that is produced primarily in neurons. In pathological cases, the monomeric form of the protein gradually forms oligomeric structures and insoluble fibrous assemblies, which are accumulated in the cell in the form of LBs. Overexpression or mutation of α-Syn leads to progressive defects and loss of dopaminergic neurons in the substantia nigra, thereby contributing to the development of Parkinson's disease. Therefore, α-Syn is clinically used as the main biomarker of Parkinson's disease.

Previous studies have reflected α-Syn abnormalities in the brains of Parkinson's patients, primarily by detecting abnormal α-Syn accumulation in peripheral fluids (cerebrospinal fluid (CSF), plasma, and saliva). Compared with the invasive cerebrospinal fluid, plasma is less costly and is a relatively non-invasive, readily available biomarker. However, by determining the concentration of total α-Syn in plasma by ELISA and other similar techniques, the determination results of the concentration of α-Syn in plasma of patients with Parkinson's disease are contradictory. The total α-Syn in plasma of patients with Parkinson's disease showed higher concentration, lower concentration or no statistically significant difference in the results compared with that of healthy people as a control group. In addition, α-Syn in an oligomeric or phosphorylated form also showed uncertain results. This difference is usually attributed to confounding factors before and during analysis (diurnal variation, gender or age dependence, and more importantly blood contamination), different techniques (enzyme-linked immunosorbent assay (ELISA), western blot, multi-analyte profiling (Luminex), and mass spectrometry), and measurement of different α-Syn species (total, aggregated, and exosomal) in plasma. The measurement of α-Syn in saliva is also an attractive method for biomarker assessment. It is simple and non-invasive to collect and has no possible blood contamination. However, the total protein content of α-Syn measured in saliva is much lower than that measured in plasma, and the protein concentration may vary throughout the day. Due to the different types of materials used for protein enrichment, α-Syn may not be sufficiently enriched. Meanwhile, the level of α-Syn in saliva is largely influenced by other proteins, including lipids and protein hydrolases (present in saliva). Those two factors cause the determination results of α-Syn in saliva to differ greatly from the actual results. Therefore, the development of a non-invasive, sensitive and effective method for diagnosis of Parkinson's disease has become an urgent problem to be solved.

Oral bioprobe delivery is considered to be a promising method for non-invasive diagnosis of diseases. The reason is primarily that the oral delivery provides the convenience to use anywhere for patients, and does not cause pain to the patient, thus achieving high compliance; and that the oral bioprobe does not require a strict sterilization process, has relatively low production costs, and is relatively inexpensive. Therefore, those who can receive the oral administration do not prefer the injectable administration. In the oral administration process, it is necessary to overcome the strong acidic pH (between 1.0 and 3.0) of the stomach; meanwhile, the gastrointestinal tract contains a large number of protein hydrolases and deoxyribonucleases, which contributes to the hindrance in developing oral bioprobes. In order solve the above problems, a pH-responsive capsule is most commonly used for delivery of biomacromolecules. The capsule can effectively protect biomacromolecules from strong acid of stomach and the degradation of a large number of enzymes in stomach in the transgastric process. However, the capsule begins to disintegrate in the more neutral environment of the intestinal tract to release the biomacromolecules. Although this method can effectively overcome the influence of strong acid of stomach on biomacromolecules, it cannot overcome the degradation of biomacromolecules by a large amount of degrading enzymes in the intestinal tract.

The luminescent functional metal organic framework (L-MOF) is a crystalline porous material formed by rare earth metal ions as the center and organic small molecules as the ligand under certain conditions. The fluorescence of the luminescent functional metal organic framework is mainly caused by emitting the sharp but weak transition forbidden by the electric dipole selection rule, and the luminescent intensity is increased through the antenna effect. The porous luminescent functional metal organic framework finds application in many areas, particularly in medical imaging. In recent years, it has been studied that the pore size of metal organic frameworks can be accurately combined with the size of DNA. Their surface charge and pore size can be tailored to effectively embed DNA. The pore size through L-MOF is just enough to load DNA. Since the three-dimensional structure loaded with DNA is limited by the pore size of the luminescent functional metal organic framework, the biomacromolecule is effectively protected under strong acids. Meanwhile, due to the design of the pore size, there is no way to allow DNA hydrolases or protein hydrolases to enter. Therefore, the DNA hydrolases have no means to degrade the DNA loaded into the cavity of the luminescent functional metal organic framework.

Based on the above background, the acid-resistant luminescent metal organic framework provides a good carrier for the design of oral bioprobes. That is, physically immobilizing DNA in the pore to form an armor that effectively protects its encapsulated DNA under extreme gastrointestinal (GI) conditions. However, how to detect the α-Syn causing Parkinson's disease in the intestinal tract specifically and effectively remains unresolved.

In this regard, aptamer, a biomaterial with good specificity, simple synthesis and stability, has attracted our attention. The aptamer is a single-stranded oligonucleotide that can bind to a target with high specificity and high affinity as a result of screening from a single-stranded random oligonucleotide library by systematic evolution of ligands by exponential enrichment (SELEX). Compared with the traditional biological recognition element antibodies, it has more superior properties, such as strong affinity and high selectivity in recognizing target molecules, easy synthesis, easy labeling, and stable properties (not damaged at 37° C.). Based on the unique superiority, the aptamer has become a new generation of biological recognition element with high specificity and high stability. However, due to the low level of α-Syn in the intestinal tract and the serious interference of intestinal matrix, there is a need for a detection means that must be able to identify with high specificity and an effective method for deducting the matrix interference.

### SUMMARY

An object of the present invention is to provide a bioprobe for non-invasive diagnosis of Parkinson's disease triggered by an intestinal microenvironment, in particular to a bioprobe of a luminescent metal organic framework-gold-aptamer complex for the detection of α-Syn as an important diagnosis marker of Parkinson's disease.

In order to achieve the above object, the present invention provides the following technical schemes.

The present invention provides a preparation method for a bioprobe for non-invasive diagnosis of Parkinson's disease triggered by an intestinal microenvironment, which comprises the following steps:
(1) mixing europium nitrate with an organic ligand, and synthesizing a luminescent metal organic framework by a solvothermal method;
(2) subjecting gold nanoparticles to a mixing reaction with aptamers to obtain Au-aptamer complexes; and
(3) dissolving the luminescent metal organic framework in an Au-aptamer complex solution for a reaction, and washing the reaction product sequentially with deionized water, an ethanol solution and a sodium dodecyl sulfate solution after the reaction, to obtain a luminescent metal organic framework adsorbing Au-aptamer complexes, namely the bioprobe.

Preferably, the europium nitrate and the organic ligand are in a molar ratio of 8-12:1. Preferably, the organic ligand comprises one of 13,3″′-dihydroxy-2′,2″,5′,5″-tetramethyl-[1,1′:4′,1″:4″,1″′-quaterphenyl]-4,4″′-dicarboxylic acid, [1,1′:4′,1″:4″,1″′-quaterphenyl]-4,4″′-dicarboxylic acid and [1,1′:4′,1″:4″,1″′-tetraphenyl]-3,3″′,5,5″′-tetracarboxylic acid.

Preferably, the solvothermal method is performed at a temperature of 160-200° C. for a period of 10-14 h.

Preferably, the mixing reaction is performed at a temperature of 25-37° C. at a rotation speed of 200-1000 rpm for a period of 3-5 h.

Preferably, the Au-aptamer complex solution is prepared with ultrapure water, and the Au-aptamer complex solution has a concentration of 0.5-5 mg/mL; the luminescent metal organic framework and the Au-aptamer complex solution are in a mass-to-volume ratio of 8-12 mg:8-12 mL.

Preferably, the reaction in the step (3) is performed at a temperature of 25-37° C. for a period of 4-6 h.

The present invention also provides a bioprobe for non-invasive diagnosis of Parkinson's disease triggered by an intestinal microenvironment obtained by the preparation method.

The present invention also provides use of the bioprobe in preparing a medicament for non-invasive diagnosis of Parkinson's disease triggered by an intestinal microenvironment.

The present invention also provides use of the detection reagent in preparing a medicament for non-invasive detection of intestinal α-synuclein triggered by an intestinal microenvironment.

The bioprobe provided by the present invention is based on the loading of aptamers modified on the surface of gold nanoparticles (Au-Aptamers) into an acid-resistant luminescent metal organic framework by accurately controlling the pore size. After the bioprobe is formed, the fluorescence emitted by the luminescent metal organic framework is absorbed by visible light of Au-Aptamers on the probe through fluorescence resonance energy transfer, so that the fluorescence of the luminescent metal organic framework at 545 nm is quenched. The pore size (diameter of approximately 35 Å) of the luminescent metal organic framework material allows aptamers (22-26 Å) on the surface of Au nanoparticles to be encapsulated into the pores of the luminescent metal framework through interaction of hydrophilic or electrostatic forces, but does not allow DNA hydrolases (DNase, 51.55 Å, 61.07 Å, 104.47 Å) to enter the pores of the framework, so that the aptamers are protected from denaturation and inactivation or hydrolysis.

The mechanism of the non-invasive diagnosis of Parkinson's disease triggered by the intestinal microenvironment of the bioprobe provided by the present invention is as follows: when a patient with Parkinson's disease is orally administered with the bioprobe, due to the presence of α-Syn in the intestinal microenvironment of the patient, the α-Syn can be specifically identified by the aptamer in the bioprobe to form an Au-Aptamer/α-Syn complex, and the Au-Aptamer/α-Syn complex is released from the luminescent metal organic framework to the intestinal tract, so that the fluorescent signal in the probe is changed from an “turn-off” state to an “turn-on” state. The “turn-on” process of fluorescence in situ can be monitored by an in vivo imager. In addition, the luminescent metal organic framework remains intact along the intestinal tract and is excreted through the feces. Therefore, the Parkinson's disease can be effectively diagnosed by quantitatively detecting the fluorescence intensity of the feces, so that the non-invasive, specific and effective diagnosis of Parkinson's disease is realized. The method provides a solution for non-invasive diagnosis of Parkinson's disease and opens up a new approach for early diagnosis and potential treatment of Parkinson's disease.

### BRIEF DESCRIPTION OF THE DRAWINGS

FIGS. 1A-1B show TEM images of a europium-based acid-resistant luminescent metal organic framework and a bioprobe prepared in the examples;

FIGS. 2A-2B show confocal micrographs of a europium-based acid-resistant luminescent metal organic framework and a bioprobe prepared in Example 1;

FIG. 3 shows fluorescence spectra of α-Syn at different concentrations in vitro;

FIG. 4 shows a standard curve of fluorescence intensity at 545 nm of α-Syn at different concentrations in vitro;

FIGS. 5A-5B show fluorescence signals in the gastrointestinal tract of a Parkinson's disease mouse model, in which FIG. 5A shows mouse imaging, and FIG. 5B shows mouse intestinal tract imaging.

### DETAILED DESCRIPTION

The present invention provides a preparation method for a bioprobe for non-invasive diagnosis of Parkinson's disease triggered by an intestinal microenvironment, which comprises the following steps:
(1) mixing europium nitrate with an organic ligand, and synthesizing a luminescent metal organic framework by a solvothermal method;
(2) subjecting gold nanoparticles to a mixing reaction with aptamers to obtain Au-aptamer complexes; and
(3) dissolving the luminescent metal organic framework in an Au-aptamer complex solution for a reaction, and washing the reaction product sequentially with deionized water, an ethanol solution and a sodium dodecyl sulfate solution after the reaction, to obtain a luminescent metal organic framework adsorbing Au-aptamer complexes, namely the bioprobe.

In the present invention, when a bioprobe for non-invasive diagnosis of Parkinson's disease triggered by an intestinal microenvironment is prepared, europium nitrate is mixed with an organic ligand, and a luminescent metal organic framework is synthesized by a solvothermal method.

In the present invention, the europium nitrate and the organic ligand are preferably in a molar ratio of 8-12:1, and further preferably 10:1.

In the present invention, the organic ligand is preferably one of 13,3″′-dihydroxy-2′,2″,5′,5″-tetramethyl-[1,1′:4′,1″:4″,1″′-quaterphenyl]-4,4″′-dicarboxylic acid, [1,1′:4′,1″:4″,1″′-quaterphenyl]-4,4″′-dicarboxylic acid and [1,1′:4′,1″:4″,1″′-tetraphenyl]-3,3″′,5,5″′-tetracarboxylic acid, and further preferably 13,3″′-dihydroxy-2′,2″,5′,5″-tetramethyl-[1,1′:4′,1″:4″,1″′-quaterphenyl]-4,4″′-dicarboxylic acid.

In the present invention, the europium nitrate and the organic ligand are mixed in a DMF (N,N-dimethylformamide) solvent.

In the present invention, the europium nitrate, the organic ligand and the DMF are preferably in a molar-to-volume ratio of 8-12 mmol:1 mmol:8-12 mL, and further preferably 10 mmol:1 mmol:10 mL.

In the present invention, when the europium nitrate is mixed with the organic ligand, ultrasonic mixing is preferably adopted.

In the present invention, the ultrasonic mixing is preferably performed at a power of 100-1500 W, and further preferably 500 W.

In the present invention, the ultrasonic mixing is preferably performed for a period of 0.5-1.5 min, and further preferably 1 min.

In the present invention, the europium nitrate and the organic ligand are preferably synthesized in a 50 mL Teflon-lined stainless steel autoclave.

In the present invention, the solvothermal method is preferably performed at a temperature of 160-200° C., and further preferably 180° C.

In the present invention, the solvothermal method is preferably performed for a period of 10-14 h, and further preferably 12 h.

In the present invention, the luminescent metal organic framework preferably has an acid resistance range of pH 1.0-3.0, and further preferably pH 2.0; and preferably has a pore size of 30-40 Å, and further preferably 35 Å.

In the present invention, gold nanoparticles are subjected to a mixing reaction with aptamers to obtain Au-aptamer complexes.

In the present invention, the gold nanoparticles are preferably prepared as follows: an aqueous solution (2.5 mL) of 1 wt % trisodium citrate is added to a boiling aqueous solution (100 mL) containing 0.01 wt % HAuCl₄, and immediately after addition, the solution is rapidly rotated (1000 rpm) in a flask, until the color of the solution gradually changes from gray to blue and then from purple to wine-red. Thereafter, the solution is allowed to boil under vigorous stirring (1000 rpm) for 10 min to verify the completion of the reaction. Finally, the solution is cooled to an ambient temperature (25° C.) and then stored at 4° C. for later use.

In the present invention, the aptamers are preferably aptamers of α-Syn, with a nucleotide sequence of 5′-SH-TTTTTGGTGGCTGGAGGGGGCGCGAACG, as shown in SEQ ID NO: 1, purchased from Sangon Biotech (Shanghai, China, https://www.sangon.com/). The purchased aptamer has been subjected to a sulfhydrylation treatment.

In the present invention, the gold nanoparticles and the aptamers are preferably in a molar concentration ratio of 0.5-1.5:1.5-2.5, and further preferably 1:2.

In the present invention, the mixing reaction of the gold nanoparticles and the aptamers is preferably performed at a temperature of 25-37° C., and further preferably 37° C.

In the present invention, the mixing reaction of the gold nanoparticles and the aptamers is preferably performed at a rotation speed of 200-1000 rpm, and further preferably 300 rpm.

In the present invention, the mixing reaction of the gold nanoparticles and the aptamers is preferably performed for a period of 3-5 h, and further preferably 4 h.

In the present invention, after the completion of the mixing reaction, the Au-aptamer complexes are further preferably washed.

In the present invention, the washing is preferably performed with a sodium dodecyl sulfate solution to remove unreacted aptamers.

In the present invention, the washing is preferably performed 2-4 times, and further preferably 3 times.

In the present invention, after each washing, centrifugal separation is preferably performed, followed by lyophilization at −40° C. to obtain Au-aptamer complexes.

In the present invention, the centrifugal separation is preferably performed at a rotation speed of 8000-12,000 rpm, and further preferably 10,000 rpm.

In the present invention, the centrifugal separation is preferably performed for a period of 8-12 min, and further preferably 10 min.

In the present invention, the luminescent metal organic framework prepared above is dissolved in an Au-aptamer complex solution for a reaction, and the reaction product is washed sequentially with deionized water, an ethanol solution and a sodium dodecyl sulfate solution to obtain a luminescent metal organic framework adsorbing Au-aptamer complexes, namely the bioprobe.

In the present invention, the Au-aptamer complex solution is preferably prepared with ultrapure water.

In the present invention, the Au-aptamer complex solution preferably has a concentration of 0.5-5 mg/mL, and further preferably 1 mg/mL.

In the present invention, the luminescent metal organic framework and the Au-aptamer complex solution are preferably in a mass-to-volume ratio of 8-12 mg:8-12 mL, and further preferably 10 mg:10 mL.

In the present invention, the reaction is preferably performed at a temperature of 25-37° C., and further preferably 37° C.

In the present invention, the reaction is preferably performed for a period of 4-6 h, and further preferably 5 h.

In the present invention, the ethanol solution preferably has a volume concentration of 70%.

In the present invention, the sodium dodecyl sulfate solution preferably has a concentration of 5% (W/W).

In the present invention, the washing is preferably performed for each reagent 3-5 times independently, and further preferably 4 times independently.

In the present invention, after the completion of the washing, the luminescent metal organic framework adsorbing Au-aptamer complexes is separated.

In the present invention, the separation is preferably centrifugal separation, followed by lyophilization at −40° C.

In the present invention, the centrifugal separation is preferably performed at a rotation speed of 8000-12,000 rpm, and further preferably 10,000 rpm.

In the present invention, the centrifugal separation is preferably performed for a period of 8-12 min, and further preferably 10 min.

The present invention also provides a bioprobe for non-invasive diagnosis of Parkinson's disease triggered by an intestinal microenvironment obtained by the preparation method.

The present invention also provides use of the bioprobe in preparing a medicament for non-invasive diagnosis of Parkinson's disease triggered by an intestinal microenvironment.

The present invention also provides use of the detection reagent in preparing a medicament for non-invasive detection of intestinal α-synuclein triggered by an intestinal microenvironment.

The technical schemes provided by the present invention will be described in detail below with reference to the examples, which, however, should not be construed as limiting the scope of the present invention.

### Example 1

(1) Preparation of a porous acid-resistant luminescent metal organic framework: Eu(N03)₃ (136 mg, 0.3 mmol) and 13,3″′-dihydroxy-2′,2″,5′,5″-tetramethyl-[1,1′:4′,1″:4″,1″′-tetrabiphenyl]-4,4″′-dicarboxylic acid (43.5 mg, 0.03 mmol) were dispersed in 10 mL of DMF, and the suspension was sonicated at 500 W for 1 min. The resulting solution was then transferred to a 50 mL Teflon-lined stainless steel autoclave and subjected to a heat treatment at 180° C. for 12 h, so that a luminescent metal organic framework with porous and acid resistant properties was obtained. The obtained luminescent metal organic framework was dried in a glove box at 80° C. for 4 h. The luminescent metal organic framework was determined to have an acid resistance value of pH 1.0-3.0 and a pore size of 35 Å.
(2) Preparation of Au-aptamers: an aqueous solution (2.5 mL) of 1 wt % trisodium citrate was added to a boiling aqueous solution (100 mL) containing 0.01 wt % HAuCl₄, and immediately after addition, the solution was rapidly rotated (1000 rpm) in a flask, until the color of the solution gradually changed from gray to blue and then from purple to wine-red. Thereafter, the solution was allowed to boil under vigorous stirring (1000 rpm) for 10 min to verify completion of the reaction, so that gold nanoparticles were obtained. Finally, the solution was cooled to an ambient temperature (25° C.) and then stored at 4° C. for later use.

The aptamers were aptamers of α-Syn, with a nucleotide sequence of 5′-SH-TTTTTGGTGGCTGGAGGGGGCGCGAACG, as shown in SEQ ID NO: 1, purchased from Sangon Biotech (Shanghai, China, https://www.sangon.com/).

A 1 μM gold nanoparticle solution (prepared with ultrapure water) was mixed with a 2 μM aptamer solution (prepared with ultrapure water), and the mixture was slowly rotated (300 rpm) at 37° C. for 4 h, subjected to centrifugal separation (10,000 rpm, 10 min), and washed with ultrapure water 3 times, each followed by centrifugal separation (10,000 rpm, 10 min), so that unreacted aptamers were removed by washing and centrifugation. Finally, the solid obtained by the last centrifugal separation was lyophilized to obtain Au-aptamers, which were stored at 4° C. for later use.
(3) Modification of the luminescent metal organic framework with the Au-aptamers: an Au-aptamer solution at a concentration of 1 mg/mL was prepared with ultrapure water as a solvent. The dried luminescent metal organic framework (10 mg) was quickly taken out of the glove box and dissolved in 10 mL of the Au-aptamer solution at room temperature (25° C.) for 5 h of reaction. Then, the mixture was centrifuged (10,000 rpm, 10 min), washed sequentially with deionized water, a 70% ethanol solution and a 5% (W/W) sodium dodecyl sulfate solution, each for 4 times (SDS can remove aptamers adsorbed on the surface of the luminescent metal organic framework so as to only reserve aptamers inside its pores), and subjected to centrifugal separation (10,000 rpm, 10 min), so that the bioprobe with the Au-aptamers adsorbed inside the luminescent metal organic framework.

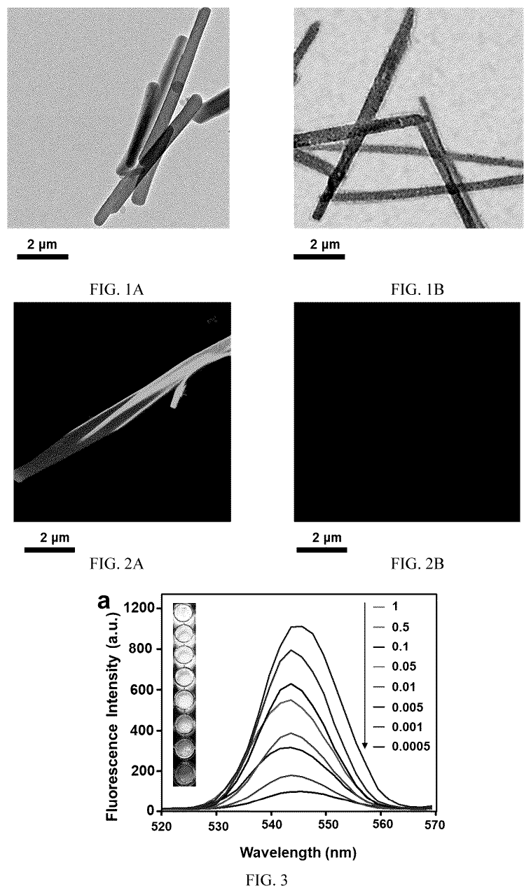

The luminescent metal organic framework prepared in the step (1) exhibited a one-dimensional linear structure by TEM, as shown in FIG. 1A. After the modification with the Au-aptamers, as shown in FIG. 1B, individually separated gold nanoparticles can be seen clearly in the luminescent metal organic framework from FIG. 1B, suggesting that the Au-aptamers can be accurately loaded into the cavity of the luminescent metal organic framework to form a bioprobe. FIGS. 2A-2B show confocal micrographs of the luminescent metal organic framework (FIG. 2A) and the bioprobe (FIG. 2B). It shows that the luminescent metal organic framework can exhibit green fluorescence. However, when the aptamers modified on the surface of the gold nanoparticles were effectively loaded into the cavity of the luminescent metal organic framework, the green fluorescence exhibited by the luminescent metal organic framework was quenched through fluorescence resonance energy transfer of the gold nanoparticles, so that the non-luminescent bioprobe was obtained. This further confirms the formation of the bioprobe.

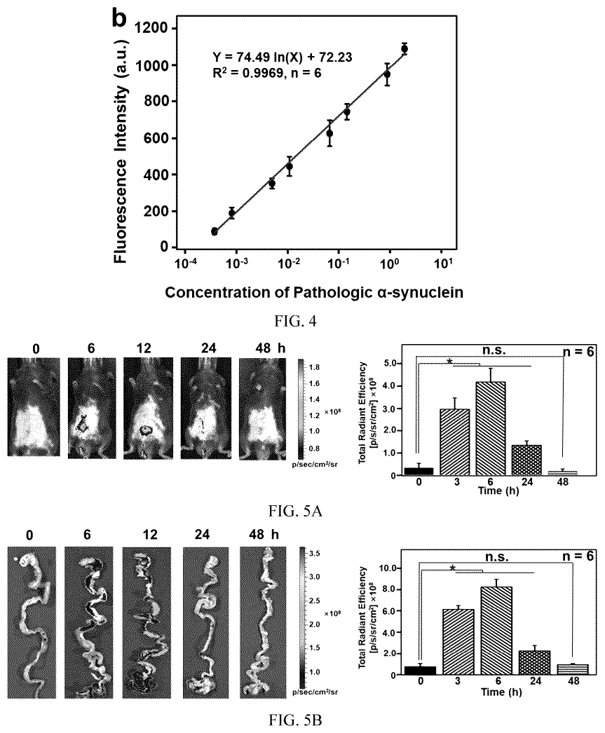

The bioprobe prepared above was used for in vitro detection and the fluorescence spectra and a standard curve of α-Syn at different concentrations were plotted. α-Syn at different concentrations was dissolved in PBS to react with 1 mg/mL bioprobe for 30 min. The solution after the reaction was measured for its fluorescence intensity by the FL-4600 molecular fluorometer. The results are shown in FIGS. 3 and 4. As can be seen from the figures, the bioprobe provided by the present invention can realize an in vitro α-Syn detection with high detection sensitivity and good standard curve linearity.

The present invention also constructs a Parkinson's disease mouse model for an in vivo experiment.

Construction of Parkinson's Disease Mouse Model:

MPTP (1-methyl-4-phenyl-1,2,3,6-tetrahydropyridine) has high lipid solubility, is easy to permeate the blood-brain barrier, and can be converted into an effective component MPP⁺ under the action of glial cell monoamine oxidase after entering the brain. MPP⁺ can inhibit the activity of mitochondrial complex I and lead to the degeneration and death of dopaminergic neurons after it is taken into the mitochondria of dopaminergic neurons by dopamine transporters.

8-week-old male mice C57BL/6 were intraperitoneally injected with MPTP (0.6 mg, 2 mg/mL) daily for 7 consecutive days to obtain mice with Parkinson's disease.

Then, in the mice with Parkinson's disease, after oral administration of the bioprobe (at a dose of 50 mg/kg), fluorescence signals to the gastrointestinal tract were detected by a small animal imager, as shown in FIGS. 5A-5B. This suggests that the bioprobe triggered by the intestinal microenvironment can respond to the α-Syn of the gastrointestinal tract. Meanwhile, the bioprobe triggered by the intestinal microenvironment can be retained along with the feces. The content of α-Syn in the feces of mice was quantitatively determined and compared with that obtained using a commercial ELISA kit, and the results are shown in Table 1.

TABLE 1
Comparison of the results of α-Syn concentration in feces obtained using
the bioprobe of Example 1 vs. a commercial ELISA kit (x̅ ± s, n = 6)

| | | | Enzyme-linked | | |
|---|---|---|---|---|---|
| | | Additional | immunosorbent | | |
| | Background | concentration | assay kit | Bioprobe | Recovery |
| Sample | (μg/mL) | (μg/mL) | (μg/mL) | (μg/mL) | (%) |
| 1 | 2.0 | 0.050 | 2.059 ± 0.011 | 2.051 ± 0.001 | 102.00 |
| 2 | 0.4 | 0.100 | 0.495 ± 0.011 | 0.498 ± 0.009 | 98.00 |
| 3 | 0 | 0.500 | 0.490 ± 0.050 | 0.493 ± 0.091 | 98.60 |
| 4 | 0 | 1.000 | 0.980 ± 0.040 | 0.982 ± 0.060 | 98.20 |
| 5 | 0.3 | 5.000 | 5.310 ± 0.250 | 5.310 ± 0.150 | 100.20 |
| 6 | 5 | 10.000 | 14.508 ± 0.312 | 14.489 ± 0.249 | 94.89 |

As can be seen from Table 1, the detection result of the developed non-invasive oral bioprobe for the intestinal microenvironment on the feces of mice with Parkinson's disease was consistent with the result obtained by the commercial enzyme-linked immunosorbent assay kit, demonstrating the reliability of the results.

According to the above examples, the present invention provides a non-invasive oral bioprobe based on an intestinal microenvironment, for the purpose of diagnosis of Parkinson's disease at an early stage. This provides a brand new scheme for oral bioprobes, and solves the problem that the oral bioprobes cannot be stably present in the gastrointestinal tract. Moreover, the method is suitable for patients to test themselves at home, thereby bringing great convenience to the patients. The method opens up a new approach for early non-invasive diseases.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A preparation method for a bioprobe for a non-invasive diagnosis of Parkinson's disease triggered by an intestinal microenvironment, comprising the following steps:
(1) mixing europium nitrate with an organic ligand, and synthesizing a luminescent metal organic framework by a solvothermal method;
(2) subjecting gold nanoparticles to a mixing reaction with aptamers to obtain Au-aptamer complexes; and
(3) dissolving the luminescent metal organic framework in an Au-aptamer complex solution for a reaction to obtain a reaction product, and washing the reaction product sequentially with deionized water, an ethanol solution, and a sodium dodecyl sulfate solution after the reaction to obtain a luminescent metal organic framework adsorbing the Au-aptamer complexes, wherein the luminescent metal organic framework adsorbing the Au-aptamer complexes is the bioprobe.

2. The preparation method according to claim 1, wherein the europium nitrate and the organic ligand are in a molar ratio of (8-12):1.

3. The preparation method according to claim 2, wherein the organic ligand is one selected from the group consisting of 13,3″′-dihydroxy-2′,2″,5′,5″′-tetramethyl-[1,1′:4′,1″:4″,1″′-quaterphenyl]-4,4″′-dicarboxylic acid, [1,1′:4′,1″:4″,1″′-quaterphenyl]-4,4″′-dicarboxylic acid, and [1,1′:4′,1″:4″,1″′-tetraphenyl]-3,3″′,5,5″′-tetracarboxylic acid.

4. The preparation method according to claim 3, wherein the solvothermal method is performed at a temperature of 160-200° C. for a period of 10-14 h.

5. The preparation method according to claim 4, wherein the mixing reaction is performed at a temperature of 25-37° C. at a rotation speed of 200-1000 rpm for a period of 3-5 h.

6. The preparation method according to claim 5, wherein the Au-aptamer complex solution is prepared with ultrapure water, and the Au-aptamer complex solution has a concentration of 0.5-5 mg/mL; the luminescent metal organic framework and the Au-aptamer complex solution are in a mass-to-volume ratio of (8-12) mg:(8-12) mL.

7. The preparation method according to claim 6, wherein the reaction in the step (3) is performed at a temperature of 25-37° C. for a period of 4-6 h.

8. A bioprobe for a non-invasive diagnosis of Parkinson's disease triggered by an intestinal microenvironment obtained by the preparation method according to claim 1.

9. A method of use of the bioprobe according to claim 8 in preparing a medicament for the non-invasive diagnosis of Parkinson's disease triggered by the intestinal microenvironment.

10. A method of use of the bioprobe according to claim 8 in preparing a medicament for a non-invasive detection of intestinal α-synuclein triggered by an intestinal microenvironment.

11. The bioprobe according to claim 8, wherein in a process of preparing the bioprobe, the europium nitrate and the organic ligand are in a molar ratio of (8-12):1.

12. The bioprobe according to claim 11, wherein in the process of preparing the bioprobe, the organic ligand is one selected from the group consisting of 13,3″′-dihydroxy-2′,2″,5′,5″′-tetramethyl-[1,1′:4′,1″:4″,1″′-quaterphenyl]-4,4″′-dicarboxylic acid, [1,1′:4′,1″:4″,1″′-quaterphenyl]-4,4″′-dicarboxylic acid, and [1,1′:4′,1″:4″,1″′-tetraphenyl]-3,3″′,5,5″′-tetracarboxylic acid.

13. The bioprobe according to claim 12, wherein in the process of preparing the bioprobe, the solvothermal method is performed at a temperature of 160-200° C. for a period of 10-14 h.

14. The bioprobe according to claim 13, wherein in the process of preparing the bioprobe, the mixing reaction is performed at a temperature of 25-37° C. at a rotation speed of 200-1000 rpm for a period of 3-5 h.

15. The bioprobe according to claim 14, wherein in the process of preparing the bioprobe, the Au-aptamer complex solution is prepared with ultrapure water, and the Au-aptamer complex solution has a concentration of 0.5-5 mg/mL; the luminescent metal organic framework and the Au-aptamer complex solution are in a mass-to-volume ratio of (8-12) mg:(8-12) mL.

16. The bioprobe according to claim 15, wherein in the process of preparing the bioprobe, the reaction in the step (3) is performed at a temperature of 25-37° C. for a period of 4-6 h.
